(19) United States
(12) Patent Application Publication
(10) Pub. No.: US 2026/0357169 A1
(43) Pub. Date: Nov. 12, 2026

(54) REACTION APPARATUS AND METHOD FOR PRODUCING PRODUCT

(71) Applicant: Panasonic Intellectual Property Management Co., Ltd., Osaka (JP)

(72) Inventors: TAKASHI KANNO, Osaka (JP); FUMIYA WAYAMA, Osaka (JP); DAISUKE KOYA, Osaka (JP); JUNKO MURAOKA, Kyoto (JP); NORIYUKI HATSUGAI, Kyoto (JP)

(21) Appl. No.: 19/717,716

(22) Filed: Jun. 24, 2026

Related U.S. Application Data
(63) Continuation of application No. PCT/JP2024/045395, filed on Dec. 23, 2024.

(30) Foreign Application Priority Data
Jan. 17, 2024 (JP) 2024-005350

(51) Int. Cl. C12M 1/40; C12M 1/00; C12N 9/04; C12N 11/091; C12P 19/02

(52) U.S. Cl. CPC C12M 21/18; C12M 23/58; C12M 29/04; C12N 9/0006; C12N 11/091; C12P 19/02; C12Y 101/01

(57) ABSTRACT

A reaction apparatus according to the present disclosure includes an enzyme reactor, an enzyme, and an electrochemical reactor. The enzyme is disposed inside the enzyme reactor. In the reaction apparatus, the enzyme reactor and the electrochemical reactor are configured to circulate a reaction solution between the enzyme reactor and the electrochemical reactor. The reaction solution contains a substrate and a coenzyme. The substrate is oxidized or reduced by the enzyme. The coenzyme restores activity of the enzyme after the enzyme reacts with the substrate.

### BACKGROUND

### 1. Technical Field

The present disclosure relates to a reaction apparatus and a method for producing a product.

### 2. Description of the Related Art

Enzymes have traditionally been known as one of the catalysts in living organisms, and substrates are converted into products that are chemically different substances through enzyme catalysis.

An example of an enzymatic reaction is an enzymatic reaction using glucose dehydrogenase. The enzymatic reaction requires glucose as a substrate and nicotinamide adenine dinucleotide (NAD) as a coenzyme. This enzymatic reaction converts the substrate into gluconolactone and the coenzyme into reduced nicotinamide adenine dinucleotide (NADH).

For example, International Publication No. 2023/074453 describes an electrochemical reaction apparatus that includes a first tank in which glucose dehydrogenase and NAD are accommodated and a working electrode is disposed and a second tank in which a counter electrode is disposed. In this electrochemical reaction apparatus, glucose dehydrogenase functions as a catalyst and NAD is involved as a coenzyme to cause the enzymatic reaction. In addition, when a voltage is applied between the working electrode and the counter electrode, NADH generated by the enzymatic reaction is oxidized, generating oxidized nicotinamide adenine dinucleotide (NAD⁺). As above, by applying a voltage between the working electrode and the counter electrode, NAD is regenerated, allowing glucose decomposition to continue even when the solution in the first tank does not contain a large amount of NAD.

Japanese Unexamined Patent Application Publication No. 60-27380 describes an enzyme reaction apparatus. This enzyme reaction apparatus includes a cylindrical container and a separation membrane. An immobilized enzyme is supported on the inner wall surface of the cylindrical container. In the immobilized enzyme, the enzyme is immobilized on water-dispersible polymer particles. The separation membrane does not allow passage of the immobilized enzyme but does allow passage of the enzymatic reaction product.

Japanese Unexamined Patent Application Publication No. 61-205499 describes an enzyme reaction apparatus. This enzyme reaction apparatus includes a reactor, a plurality of adsorbers, a desorption solution feed system, a coenzyme feed system, and a reaction solution discharge system. The adsorbers collect the coenzyme from the reaction solution produced in the reactor. The desorption solution feed system feeds a desorption solution to the adsorbers to desorb the coenzyme from the adsorbent. This enables the coenzyme to be reused.

### SUMMARY

In one general aspect, the techniques disclosed here feature a reaction apparatus including: an enzyme reactor; an enzyme disposed inside the enzyme reactor; and an electrochemical reactor, wherein the enzyme reactor and the electrochemical reactor are configured to circulate a reaction solution between the enzyme reactor and the electrochemical reactor, the reaction solution contains a substrate that is oxidized or reduced by the enzyme, the reaction solution further contains a coenzyme that restores activity of the enzyme after the enzyme reacts with the substrate, the enzyme is arranged to stay inside the enzyme reactor when the reaction solution passes through the enzyme reactor, and the electrochemical reactor electrochemically restores activity of the coenzyme.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 schematically illustrates an example of a reaction apparatus according to an embodiment;

FIG. 2 is a flowchart indicating an example of a method for producing a product according to an embodiment;

FIG. 3 illustrates another example of a reaction apparatus according to the embodiment;

FIG. 4 illustrates still another example of a reaction apparatus according to the embodiment;

FIG. 5 illustrates still another example of a reaction apparatus according to the embodiment;

FIG. 6 illustrates still another example of a reaction apparatus according to the embodiment;

FIG. 7 illustrates still another example of a reaction apparatus according to the embodiment;

FIG. 8 illustrates still another example of a reaction apparatus according to the embodiment;

FIG. 9 illustrates still another example of a reaction apparatus according to the embodiment;

FIG. 10 is a graph indicating a relationship between absorbance and reaction time in Reference Example; and

FIG. 11 is a graph indicating concentrations of fructose before and after reaction in Example and Comparative Example.

### DETAILED DESCRIPTIONS

The techniques described in International Publication No. 2023/074453, Japanese Unexamined Patent Application Publication No. 60-27380, and Japanese Unexamined Patent Application Publication No. 61-205499 have room for reconsideration from the viewpoint of continuously activating coenzymes while suppressing enzyme degradation. In view of this, one non-limiting and exemplary embodiment provides a technique advantageous in terms of continuously activating coenzymes while suppressing enzyme degradation.

The reaction apparatus according to the present disclosure is advantageous in terms of continuously activating coenzymes while suppressing enzyme degradation.

### Underlying Knowledge Forming Basis of the Present Disclosure

As described in International Publication No. 2023/074453, the coenzyme can be regenerated by applying a voltage between the working electrode and the counter electrode, enabling the coenzyme to be used repeatedly for the enzymatic reaction. However, when the enzyme is affected by the electric field produced by the application of a voltage between the working electrode and the counter electrode, degradation of the enzyme occurs, making it difficult to use the enzyme repeatedly.

Thus, the inventors of the present disclosure have conducted extensive studies on the configuration of a reaction apparatus that is advantageous in terms of continuously activating coenzymes while suppressing enzyme degradation. As a result, it was newly found that a reaction apparatus including a predetermined enzyme reactor and a predetermined electrochemical reactor is capable of continuously activating coenzymes while suppressing enzyme degradation. The inventors have completed the reaction apparatus of the present disclosure based on this new knowledge. In this disclosure, “continuously” means “repeatedly.”

### Description of Embodiments

Hereinafter, embodiments of the present disclosure will be described with reference to the drawings. The embodiments described below are all general or specific examples. The numbers, shapes, materials, components, positions of the components, connections between the components, steps, and the order of steps in the following embodiments are examples and should not be construed as limiting of the scope of the claims. The drawings are not necessarily strictly to scale. In the drawings, the same reference numerals are assigned to the components having substantially the same configuration without duplicated or detailed explanation in some cases. In the following, terms indicating relationships between components such as parallel and perpendicular, terms indicating shapes of components such as rectangular, and numerical ranges are not strictly limited to the meanings of the terms and the ranges. The terms and the ranges may include approximation, such as variations of a few percent. These embodiments indicated as general or specific examples may be implemented using a system, a device, an integrated circuit, a computer program, a recording medium such as a computer-readable CD-ROM, or any combination of a system, a method, an integrated circuit, a computer program, and a recording medium.

### Embodiments

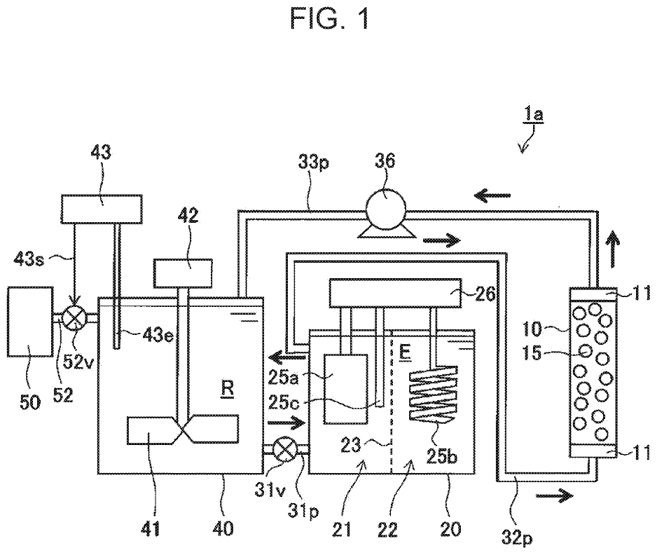

Hereinafter, embodiments will be described with reference to FIGS. 1 to 9.

FIG. 1 schematically illustrates an example of a reaction apparatus according to the embodiment. As illustrated in FIG. 1, a reaction apparatus 1a includes an enzyme reactor 10, an enzyme 15, and an electrochemical reactor 20. The enzyme 15 is disposed inside the enzyme reactor 10. In the reaction apparatus 1a, the enzyme reactor 10 and the electrochemical reactor 20 have configurations that enable circulation of a reaction solution R between the enzyme reactor 10 and the electrochemical reactor 20. The reaction solution R contains a substrate and a coenzyme. The reaction solution R may, for example, contain a solvent such as water, a buffer such as Good's buffer, a salt such as sodium chloride, and a pH adjuster such as hydrochloric acid, if necessary. The enzyme 15 is, for example, an oxidoreductase. The substrate is oxidized or reduced by the enzyme 15. The substrate may be a sugar such as fructose and glucose or may be a protein such as ovomucoid contained in egg white and casein contained in milk. The coenzyme restores the activity of the enzyme 15 after the enzyme 15 has reacted with the substrate. Examples of the coenzyme include NAD and nicotinamide adenine dinucleotide phosphate (NADP). The enzyme 15 is arranged to stay inside the enzyme reactor 10 when the reaction solution R passes through the enzyme reactor 10. The electrochemical reactor 20 electrochemically restores the activity of the coenzyme in the reaction solution R.

The circulation of the reaction solution R between the enzyme reactor 10 and the electrochemical reactor 20 allows the coenzyme in the reaction solution R to be continuously activated by the electrochemical reactor 20. In addition, the enzyme 15 is arranged to stay inside the enzyme reactor 10, so that the enzyme 15 is not affected by the electrochemical reactor 20, suppressing degradation of the enzyme 15. This enables repeated use of the enzyme 15.

As illustrated in FIG. 1, the reaction apparatus 1a includes, for example, a storage tank 40, a stirrer 41, and a motor 42. The reaction solution R is stored in the storage tank 40. The stirrer 41 is connected to the motor 42, and the motor 42 rotates the stirrer 41. The stirrer 41 is located inside the storage tank 40, and the reaction solution R is stirred by the rotation of the stirrer 41. The stirrer 41 may be configured as a magnetic stirrer.

The reaction apparatus 1a has, for example, a flow path 31p, a valve 31v, a flow path 32p, a flow path 33p, and a pump 36. The flow path 31p connects the storage tank 40 and the electrochemical reactor 20. The valve 31v is located in the flow path 31p. The flow path 32p connects the electrochemical reactor 20 and the enzyme reactor 10. The flow path 33p connects the electrochemical reactor 20 and the storage tank 40. The pump 36 is located, for example, in the flow path 33p. The pump 36 may be located in the flow path 31p or the flow path 32p.

When the pump 36 operates with the valve 31v open, the reaction solution R stored in the storage tank 40 passes through the flow path 31p, the electrochemical reactor 20, the flow path 32p, the enzyme reactor 10, and the flow path 33p, and returns to the storage tank 40. In this way, the reaction solution R can circulate between the enzyme reactor 10 and the electrochemical reactor 20.

The reaction apparatus 1a has, for example, a pH meter 43, a pH measuring electrode 43e, a signal line 43s, a feeder 50, a path 52, and a valve 52v. The pH meter 43 measures the pH of the measurement target liquid based on the potential difference between the reference electrode and the glass electrode in the pH measuring electrode 43e. The pH measuring electrode 43e is immersed in the reaction solution R stored in the storage tank 40. This enables the pH of the reaction solution R to be measured by the pH meter 43.

The feeder 50 is connected to the storage tank 40 by the path 52 and feeds a pH adjuster towards the storage tank 40. The valve 52v is located in the path 52. A signal to control the opening and closing of the valve 52v is sent to the valve 52v through the signal line 43s. This controls the opening and closing of the valve 52v. Examples of the pH adjuster include hydrochloric acid, sodium hydroxide, and malic acid.

The electrochemical reactor 20 is not limited to any particular configuration as long as it can electrochemically restore the activity of the coenzyme. As illustrated in FIG. 1, the electrochemical reactor 20 has a first chamber 21, a second chamber 22, a membrane 23, and a voltage application device 26. The reaction solution R passes through the first chamber 21. For example, the flow paths 31p and 32p are connected to the first chamber 21. A working electrode 25a is disposed in the first chamber 21. A counter electrode 25b is disposed in the second chamber 22. The membrane 23 separates the first chamber 21 from the second chamber 22. The membrane 23 prevents passage of the coenzyme and the substrate contained in the reaction solution R and has ion conductivity. The voltage application device 26 is a device that applies a voltage between the working electrode 25a and the counter electrode 25b.

When a voltage is applied between the working electrode 25a and the counter electrode 25b by the voltage application device 26, an oxidation-reduction reaction occurs at the working electrode 25a and the counter electrode 25b, activating the coenzyme.

As illustrated in FIG. 1, the first and second chambers 21 and 22 accommodate an electrolyte solution E. This facilitates the occurrence of a predetermined oxidation-reduction reaction at the working electrode 25a and the counter electrode 25b. For example, NAD⁺ is reduced to produce NADH. Examples of the electrolyte used in the electrolyte solution E include sodium chloride and potassium chloride. For example, the electrolyte solution E in the second chamber 22 does not contain the substrate and the coenzyme.

The membrane 23 is not limited to any particular membrane as long as it can prevent passage of the coenzyme and the substrate and has ion conductivity. The membrane 23 contains, for example, a polymer having perfluoro side chains containing sulfonic acid groups. With this configuration, predetermined ions, such as protons, can move through the membrane 23 easily and quickly, and the oxidation-reduction reaction to activate the coenzyme can be conducted efficiently at the working electrode 25a and the counter electrode 25b.

The materials that make up the surfaces of the working electrode 25a and the counter electrode 25b are not limited to any particular material. The surfaces of the working electrode 25a and the counter electrode 25b, for example, contain a conductive material. The conductive material is, for example, a material that does not decompose upon oxidation. Examples of the conductive material include carbon materials, conductive polymers, semiconductor materials, and metallic materials. Examples of the carbon materials include carbon nanotubes, Ketjen black (registered trademark), glassy carbon, graphene, fullerene, carbon fiber, carbon fabric, and carbon aerogel. Examples of the conductive polymers include polyaniline, polyacetylene, polypyrrole, poly(3,4-ethylenedioxythiophene), poly(p-phenylenevinylene), polythiophene, and poly(p-phenylene sulfide). Examples of the semiconductor materials include silicon, germanium, indium tin oxide (ITO), titanium dioxide, copper oxide, and silver oxide. Examples of the metallic materials include gold, platinum, silver, titanium, aluminum, tungsten, copper, iron, and palladium. The working electrode 25a may be a glassy carbon electrode, and the counter electrode 25b may be a platinum electrode.

The surface area of the counter electrode 25b is larger than that of the working electrode 25a, for example.

As illustrated in FIG. 1, for example, a reference electrode 25c is disposed in the first chamber 21. The reference electrode 25c facilitates stabilization of the potential difference between the working electrode 25a and the counter electrode 25b. The reference electrode 25c is, for example, a silver/silver chloride electrode.

The enzyme reactor 10 is not limited to any particular configuration as long as the enzyme 15 is disposed inside the enzyme reactor 10 and the enzyme 15 is arranged to stay inside the enzyme reactor 10 when the reaction solution R passes through the enzyme reactor 10. The enzyme reactor 10 is, for example, a tubular reactor. The reaction solution R is fed to the enzyme reactor 10 so that it passes through the enzyme reactor 10 in the direction opposite to the direction of gravity, for example.

The enzyme reactor 10 has, for example, filters 11. The filters 11 allow passage of the coenzyme and the substrate and prevent passage of the enzyme 15 when the reaction solution R passes through the enzyme reactor 10. This allows, in the enzyme reactor 10, the enzyme 15 to stay inside the enzyme reactor 10 while allowing the coenzyme and the substrate to be removed from the enzyme reactor 10. The filter 11, for example, allows passage of the product produced by the enzymatic reaction involving the enzyme 15. This makes it easy to collect the product obtained by the enzymatic reaction.

The enzyme reactor 10 has, for example, two filters 11. The filters 11 are located, for example, at the inlet and the outlet of the enzyme reactor 10 in the flow of the reaction solution R.

The filter 11 is, for example, a porous membrane having a predetermined average pore diameter or a fiber-containing membrane. The average pore diameter of the filter 11 is not limited to a specific value as long as it allows passage of the coenzyme and the substrate and prevents passage of the enzyme 15. As described below, for example, if the enzyme 15 is supported on surfaces of carrier particles, the average pore diameter of the filter 11 is smaller than half the average diameter of the carrier particles. This makes it easier to achieve the configuration that allows passage of the coenzyme and the substrate and prevents passage of the enzyme 15. The average pore diameter of the filter 11 can be determined, for example, by a gas adsorption method.

The enzyme 15 is supported, for example, on surfaces of carrier particles. This makes it easier for the enzyme 15 to stay inside the enzyme reactor 10 when the reaction solution R passes through the enzyme reactor 10. The enzyme 15 may be located inside the enzyme reactor 10 by itself, without being supported on a carrier.

The carrier particles are not limited to any particular particle as long as they can carry the enzyme 15 on their surfaces. The carrier particles contain, for example, an organic polymer or a nonmetallic inorganic material. In this case, the activity of the enzyme 15 can be readily maintained at a high level, enabling repeated use of the enzyme 15. An example of the organic polymer is an ion exchange resin. The nonmetallic inorganic material includes an inorganic material such as glass and ceramics and does not include metals.

The average diameter of the carrier particles is not limited to a specific value. The carrier particles have an average diameter of, for example, greater than or equal to 1 micrometer and less than or equal to 5 millimeters. This makes it easier for the enzyme 15 to stay inside the enzyme reactor 10 when the reaction solution R passes through the enzyme reactor 10. In addition, the amount of the enzyme 15 filled in the enzyme reactor 10 tends to be larger. The average diameter of the carrier particles is, for example, the median diameter in a number-based particle size distribution.

Using the above reaction apparatus 1a, a product can be produced. The method for producing a product includes causing the reaction solution R containing the substrate and the coenzyme to pass through the electrochemical reactor 20 and the enzyme reactor 10. This includes continuously causing an enzymatic reaction in the enzyme reactor 10 and electrochemically restoring the activity of the coenzyme in the electrochemical reactor 20. As described above, the enzyme 15 is arranged to stay inside the enzyme reactor 10 when the reaction solution R passes through the enzyme reactor 10.

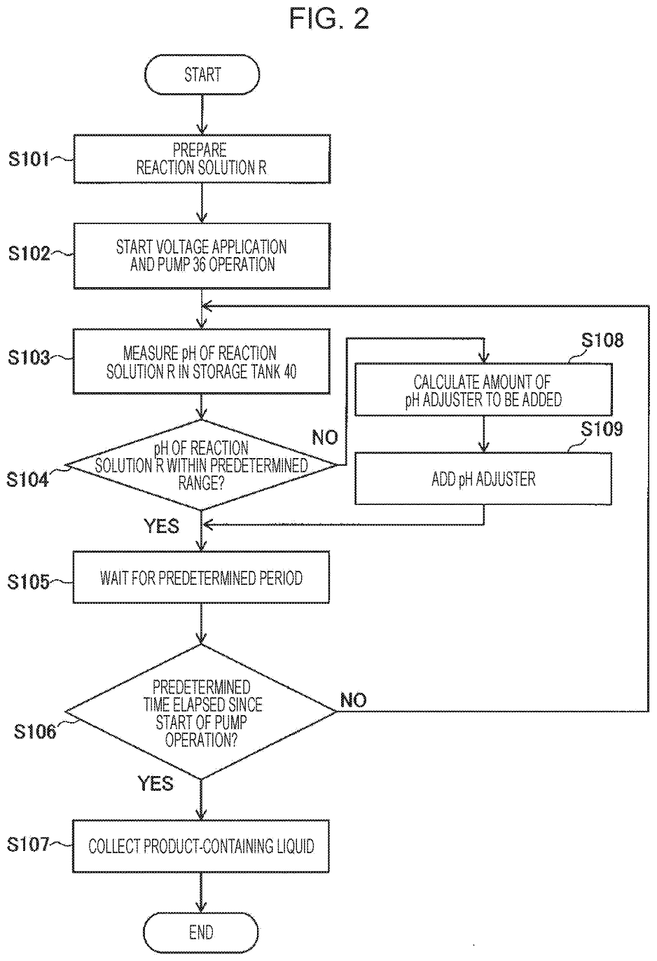

FIG. 2 is a flowchart indicating an example of a method for producing a product. As indicated in FIG. 2, the reaction solution R is prepared in step S101. For example, with the valve 31v closed, the substrate and the coenzyme are added in the storage tank 40 so as to have predetermined concentrations, and thus the reaction solution R is prepared. Next, in step S102, the valve 31v is opened, and application of a voltage between the working electrode 25a and the counter electrode 25b and operation of the pump 36 are started. This causes the reaction solution R in the storage tank 40 to pass through the electrochemical reactor 20 and the enzyme reactor 10 and return to the storage tank 40. In this way, the reaction solution R circulates between the enzyme reactor 10 and the electrochemical reactor 20.

In the enzyme reactor 10, the substrate is oxidized or reduced by the catalysis of the enzyme 15, producing a product. This converts the enzyme 15 into an inactive state, but the activity of the enzyme 15 is restored by the action of the coenzyme contained in the reaction solution R. However, the coenzyme that restored the activity of the enzyme 15 once loses its activity, but the activity of the coenzyme is electrochemically restored in the electrochemical reactor 20. Thus, the enzymatic reaction involving the enzyme 15 produces the product continuously.

Next, in step S103, the pH of the reaction solution R in the storage tank 40 is measured by the pH meter 43. Next, in step S104, it is determined whether the pH of the reaction solution R is within a predetermined range. For example, the optimal pH for the enzymatic reaction involving the enzyme 15 is within this predetermined range.

When the result of the determination in step S104 is negative, the process proceeds to step S108, where the amount of the pH adjuster to be added to the reaction solution R is calculated. In this calculation, for example, the amount of the pH adjuster to be added is determined based on the difference between the measured pH of the reaction solution R and the value corresponding to the optimal pH above. For example, if this difference is large, the amount of the pH adjuster to be added increases. Next, the process proceeds to step S109, where the pH adjuster is added. For example, a control signal to open the valve 52v is sent through the signal line 43s to open the valve 52v, and the pH adjuster is fed from the feeder 50 through the path 52 to the storage tank 40 in an amount corresponding to the calculation result obtained in step S108. In this way, the pH of the reaction solution R is adjusted to the above-described optimal pH or a value close to the optimal pH.

When the result of the determination in step S104 is positive, or when step S109 ends, the process proceeds to step S105 and, after a predetermined period of time, proceeds to step S106. The predetermined period is defined, for example, such that the pH of the reaction solution R falls within the predetermined range in about 10 minutes. It is determined whether a predetermined time (e.g., 4 hours) has elapsed since the start of operation of the pump 36. When the result of this determination is negative, the process returns to step S103. When the result of this determination is positive, the process proceeds to step S107, where the liquid containing the product is collected, and the production of the product is completed.

The series of processes indicated in FIG. 2 may be executed by automatic control using, for example, a digital computer having a memory storing a program for executing the processes and a processor that reads this program and performs operations. In this automatic control, the type of substrate, coenzyme, and the like, the volume of the reaction solution R, the type of working electrode 25a, the voltage applied between the working electrode 25a and the counter electrode 25b, the optimal pH for the enzymatic reaction involving the enzyme 15, and the overall processing time can be treated as input parameters.

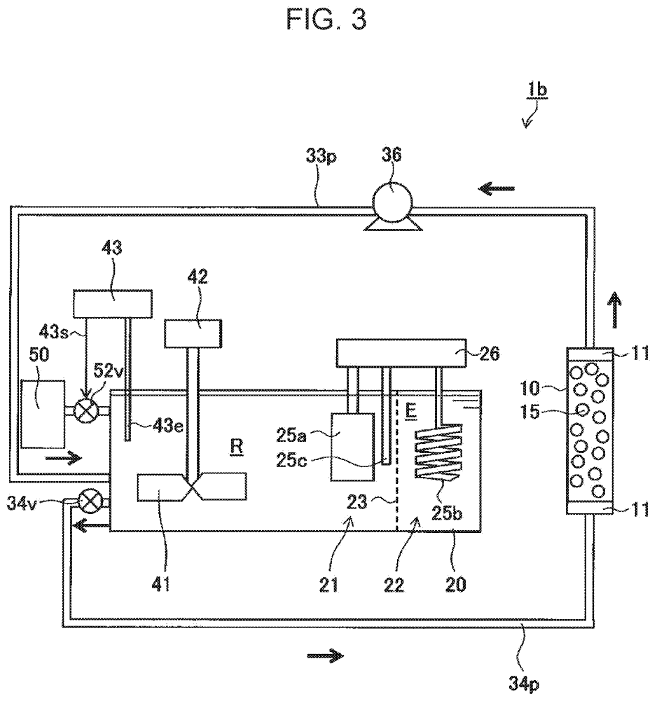

The reaction apparatus 1a can be modified from various perspectives. FIG. 3 illustrates another example of a reaction apparatus according to the embodiment. A reaction apparatus 1b illustrated in FIG. 3 has a configuration similar to that of the reaction apparatus 1a, except for portions specifically described. The components of the reaction apparatus 1b that are identical or corresponding to those of the reaction apparatus 1a are assigned the same reference numerals as those of the reaction apparatus 1a without detailed explanation. The description for the reaction apparatus 1a is also applicable to the reaction apparatus 1b without causing any technical inconsistencies.

As illustrated in FIG. 3, in the reaction apparatus 1b, the storage tank 40 and the electrochemical reactor 20 of the reaction apparatus 1a are integrated. In the first chamber 21 of the electrochemical reactor 20, in addition to the working electrode 25a, the stirrer 41 and the pH measuring electrode 43e are disposed. The reaction apparatus 1b has a flow path 34p and a valve 34v. The flow path 34p connects the first chamber 21 of the electrochemical reactor 20 and the enzyme reactor 10. The valve 34v is located in the flow path 34p.

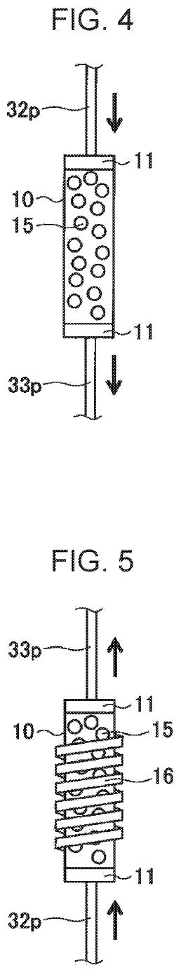

FIG. 4 illustrates still another example of a reaction apparatus according to the embodiment. As illustrated in FIG. 4, the reaction solution R may be fed to the enzyme reactor 10 so that the reaction solution R passes through the enzyme reactor 10 in the direction of gravity, for example.

FIG. 5 illustrates still another example of a reaction apparatus according to the embodiment. As illustrated in FIG. 5, a temperature controller 16 may be disposed around the enzyme reactor 10. The enzyme typically shows high activity at temperatures of 30° C. to 50° C. and can be inactivated upon prolonged exposure to environments at temperatures greater than or equal to 75° C.. Thus, it is important to keep the temperature around the enzyme 15 within a predetermined range in terms of stability of the activity of the enzyme 15 and promotion of the enzymatic reaction. Thus, the temperature controller 16 makes it easier to stabilize the activity of the enzyme 15, promoting the enzymatic reaction. The temperature controller 16 includes, for example, a coiled heat transfer tube through which a heat transfer medium such as water flows. The temperature controller 16 may include a Peltier device.

The temperature of the reaction solution R or the enzyme 15 may be regulated by placing a portion of the reaction apparatus 1a in a thermostatic chamber.

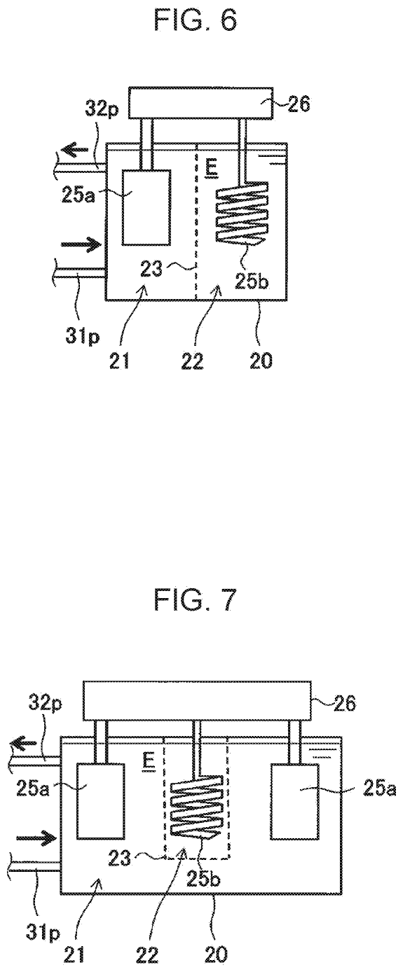

FIG. 6 illustrates still another example of a reaction apparatus according to the embodiment. As illustrated in FIG. 6, the reference electrode 25c may be omitted. If the distance between the working electrode 25a and the counter electrode 25b is small, for example within about 1 cm, the reference electrode 25c may be omitted.

FIG. 7 illustrates still another example of a reaction apparatus according to the embodiment. As illustrated in FIG. 7, the electrochemical reactor 20 may have multiple working electrodes 25a. In this case, for example, one counter electrode 25b may be placed between two working electrodes 25a. For example, when the electrochemical reactor 20 includes three working electrodes 25a, the working electrodes 25a and the counter electrodes 25b may be arranged in the order of the working electrode 25a, the counter electrode 25b, the working electrode 25a, the counter electrode 25b, and the working electrode 25a. For example, the working electrodes 25a are connected in parallel and the counter electrodes 25b are connected in parallel to the voltage application device 26.

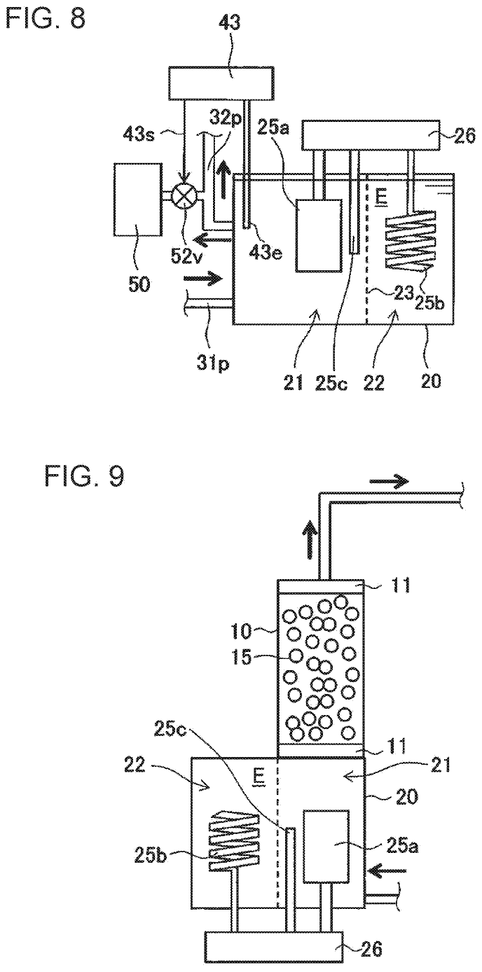

FIG. 8 illustrates still another example of a reaction apparatus according to the embodiment. In FIG. 8, the pH measuring electrode 43e is located in the first chamber 21, and the pH meter 43 measures the pH of the reaction solution R in the first chamber 21. The feeder 50 is connected to the flow path 32p and can feed the pH adjuster to the reaction solution R that flows through the first chamber 21 toward the enzyme reactor 10.

FIG. 9 illustrates still another example of a reaction apparatus according to the embodiment. As illustrated in FIG. 9, the first chamber 21 of the electrochemical reactor 20 may be directly connected to the enzyme reactor 10, and the first chamber 21 and the interior of the enzyme reactor 10 may be separated by a filter 11, for example. This configuration also allows the enzyme 15 to stay inside the enzyme reactor 10, and thus the enzyme 15 is not likely to be affected by the electrochemical reactor 20, suppressing degradation of the enzyme 15.

FIGS. 1 and 3 illustrate the overall configuration of the reaction apparatus, FIGS. 4 and 5 illustrate the configuration relating to the enzyme reactor 10, and FIGS. 6 through 9 illustrate the configuration relating to the electrochemical reactor 20. These configurations may be combined as appropriate to form a reaction apparatus.

### Other Embodiments

The reaction apparatus and the method for producing a product were described based on the embodiments. However, this disclosure is not limited to these embodiments. Without departing from the gist of the present disclosure, various changes may be added to the embodiments by a person skilled in the art, and the components in different embodiments may be combined. They are construed as being within the scope of the present disclosure.

### Appendix

The above description discloses the following techniques.

### Technique 1

A reaction apparatus including:
an enzyme reactor;
an enzyme disposed inside the enzyme reactor; and
an electrochemical reactor, wherein
the enzyme reactor and the electrochemical reactor are configured to circulate a reaction solution between the enzyme reactor and the electrochemical reactor,
the reaction solution contains a substrate that is oxidized or reduced by the enzyme,
the reaction solution further contains a coenzyme that restores activity of the enzyme after the enzyme reacts with the substrate,
the enzyme is arranged to stay inside the enzyme reactor when the reaction solution passes through the enzyme reactor, and
the electrochemical reactor electrochemically restores activity of the coenzyme.

### Technique 2

The reaction apparatus according to Technique 1, wherein the electrochemical reactor may include:
a first chamber through which the reaction solution passes and in which a working electrode is disposed;
a second chamber in which a counter electrode is disposed;
a membrane that separates the first chamber from the second chamber and that prevents passage of the coenzyme and the substrate, the membrane having ion conductivity; and
a voltage application device that applies a voltage between the working electrode and the counter electrode.

### Technique 3

The reaction apparatus according to Technique 2, wherein the first chamber and the second chamber may accommodate an electrolyte solution.

### Technique 4

The reaction apparatus according to Technique 2 or 3, wherein the membrane may contain a polymer having a perfluoro side chain containing a sulfonic acid group.

### Technique 5

The reaction apparatus according to any one of Techniques 1 to 4, further including carrier particles,
wherein the enzyme may be supported on surfaces of the carrier particles.

### Technique 6

The reaction apparatus according to Technique 5, wherein the carrier particles may each contain an organic polymer or a nonmetallic inorganic material.

### Technique 7

The reaction apparatus according to Technique 5 or 6, wherein the carrier particles may have an average diameter of greater than or equal to 1 micrometer and less than or equal to 5 millimeters.

### Technique 8

The reaction apparatus according to any one of Techniques 1 to 7, wherein the enzyme reactor may further include a filter that allows passage of the coenzyme and the substrate and that prevents passage of the enzyme when the reaction solution passes through the enzyme reactor.

### Technique 9

The reaction apparatus according to Technique 8, further including carrier particles,
wherein the enzyme may be supported on surfaces of the carrier particles, and
the filter may have an average pore diameter smaller than half of an average diameter of the carrier particles.

### Technique 10

A method for producing a product, the method including:
causing an enzymatic reaction to occur in an enzyme reactor by passing a reaction solution containing a substrate and a coenzyme through an electrochemical reactor and the enzyme reactor, the substrate being oxidized or reduced by an enzyme, the coenzyme restoring activity of the enzyme after the enzyme reacts with the substrate; and
electrochemically restoring activity of the coenzyme in the electrochemical reactor, wherein
the enzyme is arranged to stay inside the enzyme reactor when the reaction solution passes through the enzyme reactor.

### Technique 11

A method for producing a product, the method including:
causing an enzymatic reaction to occur in a reaction solution containing a substrate and a coenzyme in an enzyme reactor in which an enzyme is disposed, the substrate being oxidized or reduced by the enzyme; and
electrochemically restoring activity of the coenzyme in the reaction solution, in an electrochemical reactor, after the enzymatic reaction.

### EXAMPLES

The present disclosure will be explained in more detail by using the following examples. This disclosure is not limited to the following examples.

### Reference Example

A container was prepared that has two spaces, namely the first and second chambers, separated by a Nafion ion-exchange membrane manufactured by DuPont, similar to the electrochemical reactor 20 illustrated in FIG. 1. Nafion is a registered trademark. A working electrode was prepared by depositing gold on a glass plate having a short side length of 10 mm and a long side length of 20 mm in plan view and coating it with platinum in an aqueous platinum chloride solution. A silver/silver chloride electrode was prepared as a reference electrode. A platinum electrode was prepared as a counter electrode. The working electrode and the reference electrode were placed inside the first chamber of the container, and the counter electrode was placed inside the second chamber of the container. The working electrode, the reference electrode, and the counter electrode were electrically connected to a working electrode terminal, a reference electrode terminal, and a counter electrode terminal of a potentiostat, respectively. In the second chamber of the container, a 2-morpholinoethanesulfonic acid (MES) buffer solution was added. The MES buffer solution did not contain substances corresponding to the substrate and the coenzyme, and the pH of the MES buffer solution was 5.5. In the first chamber of the container, an MES buffer solution containing 5 mM of the inactive coenzyme NAD⁺ was added. The pH of this MES buffer solution was 5.5. The first chamber of the above container was connected to the inlet of the pump by a rubber tube, and the outlet of the pump was connected to an external container as another container by a rubber tube. In addition, the external container was connected to the first chamber of the above container by a rubber tube to form a circulation path for the MES buffer solution.

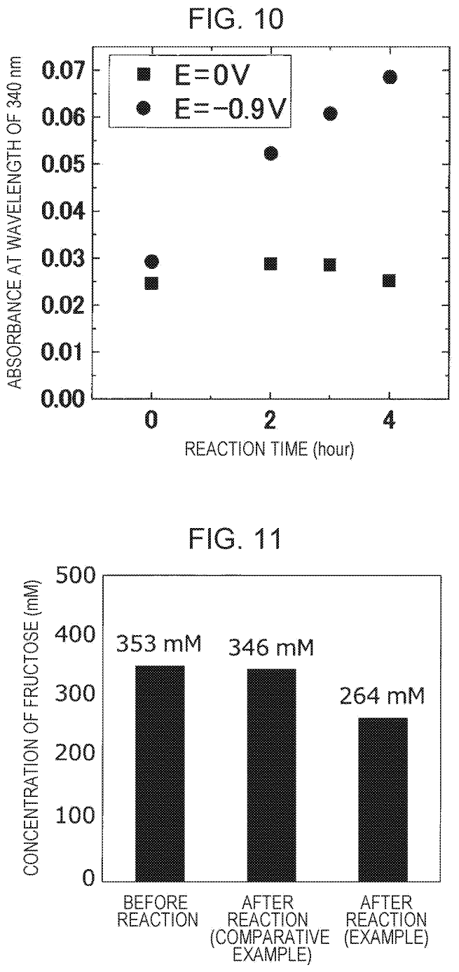

The MES buffer solution was circulated between the first chamber and the external container by operating the pump with or without a voltage applied between the working electrode and the counter electrode. Samples of the MES buffer solution circulating between the first chamber and the external container were taken at the start of pump operation, after 2 hours of pump operation, after 3 hours of pump operation, and after 4 hours of pump operation. The absorbance of these samples at a wavelength of 340 nm was measured. The results are indicated in FIG. 10.

FIG. 10 is a graph indicating a relationship between absorbance and reaction time in Reference Example. As indicated in FIG. 10, when no voltage was applied between the working electrode and the counter electrode (E=0 V), no change in absorbance was observed. In contrast, when a voltage was applied between the working electrode and the counter electrode (E=−0.9 V), the absorbance at a wavelength of 340 nm increased with time from the start of pump operation. The absorbance at a wavelength of 340 nm correlates with the concentration of NADH. Thus, it is considered that NAD⁺ is reduced and NADH is generated by the application of a voltage between the working electrode and the counter electrode. It was suggested that the activity of the coenzyme can be electrochemically restored by the application of a voltage between the working electrode and the counter electrode.

### Examples and Comparative Examples

As in Reference Example, a container having two separated spaces, namely the first and second chambers, was prepared, the working electrode and the reference electrode were placed inside the first chamber of the container, and the counter electrode was placed inside the second chamber of the container. The working electrode, the reference electrode, and the counter electrode were electrically connected to a working electrode terminal, a reference electrode terminal, and a counter electrode terminal of a potentiostat, respectively. In the second chamber of the container, a 2-morpholinoethanesulfonic acid (MES) buffer solution was added. The MES buffer solution did not contain substances corresponding to the substrate and the coenzyme, and the pH of the MES buffer solution was 5.5. In the first chamber of the container, an MES buffer solution containing 5 mM of the inactive coenzyme NAD⁺ and 350 mM of fructose was added. The pH of this MES buffer solution was 5.5.

A column filled with an enzyme-supported carrier was prepared in which mannitol dehydrogenase as the enzyme was supported on Duolite A568 ion exchange resin manufactured by DuPont. Duolite is a registered trademark of DuPont. The first chamber of the above container was connected to the inlet of the pump by a rubber tube, and the outlet of the pump was connected to the inlet of the column by a rubber tube. In addition, the outlet of the column and the first chamber of the above container were connected by a rubber tube to form a circulation path for the MES buffer solution. Porous filters each having an average pore diameter of 35 μm were located near the inlet and the outlet of the column. The filters allowed passage of the fructose and the coenzyme and prevented the passage of the enzyme-supported carrier.

The MES buffer solution was circulated between the first chamber and the column by operating the pump with or without a voltage applied between the working electrode and the counter electrode. Samples of the MES buffer solution circulating between the first chamber and the column were collected at the start of pump operation and after 24 hours of pump operation. The concentrations of fructose in these samples were determined using a fructose colorimetric assay kit available from Sigma. The results are indicated in FIG. 11.

FIG. 11 is a graph indicating the concentrations of fructose before and after the reaction in Example and Comparative Example. In Example, a voltage was applied between the working electrode and the counter electrode, and in Comparative Example, no voltage was applied between the working electrode and the counter electrode. As indicated in FIG. 11, the concentration of fructose changed little when no voltage was applied between the working electrode and the counter electrode, but the concentration of fructose decreased when a voltage was applied between the working electrode and the counter electrode. This suggested that fructose was converted into mannitol by the action of the enzyme and mannitol was formed.

The reaction apparatus of the present disclosure is applicable, for example, as a reaction apparatus using an enzymatic reaction such as conversion of fructose to mannitol.

What is claimed is:

1. A reaction apparatus comprising:
an enzyme reactor;
an enzyme disposed inside the enzyme reactor; and
an electrochemical reactor, wherein the enzyme reactor and the electrochemical reactor are configured to circulate a reaction solution between the enzyme reactor and the electrochemical reactor,
the reaction solution contains a substrate that is oxidized or reduced by the enzyme,
the reaction solution further contains a coenzyme that restores activity of the enzyme after the enzyme reacts with the substrate,
the enzyme is arranged to stay inside the enzyme reactor when the reaction solution passes through the enzyme reactor, and
the electrochemical reactor electrochemically restores activity of the coenzyme.

2. The reaction apparatus according to claim 1, wherein the electrochemical reactor includes:
a first chamber through which the reaction solution passes and in which a working electrode is disposed;
a second chamber in which a counter electrode is disposed;
a membrane that separates the first chamber from the second chamber and that prevents passage of the coenzyme and the substrate, the membrane having ion conductivity; and
a voltage application device that applies a voltage between the working electrode and the counter electrode.

3. The reaction apparatus according to claim 2, wherein the first chamber and the second chamber accommodate an electrolyte solution.

4. The reaction apparatus according to claim 2, wherein the membrane contains a polymer having a perfluoro side chain containing a sulfonic acid group.

5. The reaction apparatus according to claim 1, further comprising carrier particles,
wherein the enzyme is supported on surfaces of the carrier particles.

6. The reaction apparatus according to claim 5, wherein the carrier particles each contain an organic polymer or a nonmetallic inorganic material.

7. The reaction apparatus according to claim 5, wherein the carrier particles have an average diameter of greater than or equal to 1 micrometer and less than or equal to 5 millimeters.

8. The reaction apparatus according to claim 1, wherein the enzyme reactor further includes a filter that allows passage of the coenzyme and the substrate and that prevents passage of the enzyme when the reaction solution passes through the enzyme reactor.

9. The reaction apparatus according to claim 8, further comprising carrier particles, wherein
the enzyme is supported on surfaces of the carrier particles, and
the filter has an average pore diameter smaller than half of an average diameter of the carrier particles.

10. A method for producing a product, the method comprising:
causing an enzymatic reaction to occur in a reaction solution containing a substrate and a coenzyme in an enzyme reactor in which an enzyme is disposed, the substrate being oxidized or reduced by the enzyme; and
electrochemically restoring activity of the coenzyme in the reaction solution, in an electrochemical reactor, after the enzymatic reaction.
